(19) United States
(12) Patent Application Publication
(10) Pub. No.: US 2026/0060850 A1
(43) Pub. Date: Mar. 5, 2026

(54) SANITARY NAPKIN STRUCTURE CONVENIENT FOR ROLLING UP AFTER USE

(71) Applicant: Foshan Nicety Sanitary Products Co., Ltd., Foshan (CN)

(72) Inventors: Yana WANG, Foshan (CN); Guojie WU, Foshan (CN)

(73) Assignee: Foshan Nicety Sanitary Products Co., Ltd., Foshan (CN)

(21) Appl. No.: 19/300,612

(22) Filed: Aug. 14, 2025

(30) Foreign Application Priority Data
Aug. 28, 2024 (CN) 202422096730.8

(51) Int. Cl. A61F 13/47; A61F 13/15; A61F 13/84

(52) U.S. Cl. CPC A61F 13/4704; A61F 13/15203; A61F 13/84; A61F 2013/15243; A61F 2013/8402

(57) ABSTRACT

A sanitary napkin structure convenient for rolling up after use includes a sanitary napkin body and a foldable binding strap. A first adhesive portion is provided at a bottom surface of the sanitary napkin body. The foldable binding strap includes a peeling end, a first binding strap portion, a second binding strap portion, a first fixing portion, a second fixing portion, and a second adhesive portion, and the second fixing portion and the first fixing portion are arranged in a herringbone shape and fixed on the bottom surface of the sanitary napkin body.

### CROSS-REFERENCE TO RELATED APPLICATIONS

The present application claims priority to Chinese Patent Application No. 202422096730.8, filed on Aug. 28, 2024, the entire contents of which are incorporated herein by reference.

### TECHNICAL FIELD

The present application relates to the technical field of sanitary products, in particular to a sanitary napkin structure convenient for rolling up after use.

### BACKGROUND

At present, most of the sanitary napkin products on the market do not have a binding structure. Many users directly discard the sanitary napkins into the trash can after using them, so that the dirt on the sanitary napkins is directly exposed to the air. This is not only easy to breed bacteria, emit a strong odor, and pollute the environment, but also affects people's perception and easily causes psychological discomfort. For this reason, a sanitary napkin with a binding structure has now appeared. For example, Chinese Patent Publication CN213641592U discloses a technical solution which mainly includes a sanitary napkin body and a fixing strap. More than two adhesive portions are provided on the bottom surface of the sanitary napkin body, and are arranged along the length direction of the sanitary napkin body. The fixing strap is arranged between the two adhesive portions, the length direction of the fixing strap is also the same as the length direction of the sanitary napkin body, and the front and back of the fixing strap also have adhesive portions. When in use, the rolled up sanitary napkin body can be tied up by sticking the two ends of the fixing strap together. However, using such a structure, there are still some of the following shortcomings in actual application.

Firstly, since the back of the fixing strap needs to be bonded to the sanitary napkin body, when the fixing strap is peeled off from the sanitary napkin body, the back of the fixing strap will be stuck with the fluff of the sanitary napkin body. And since the front of the fixing strap needs to be bonded to the underwear, when the sanitary napkin body and the underwear are separated, the front of the fixing strap will be stuck with the fluff of the underwear. These fluffs will greatly reduce the adhesion of the front and back of the fixing strap, especially the fluff on the back of the fixing strap will be more, so that after the two ends of the fixing strap are pasted together, the adhesion is insufficient and it is easy to be stretched apart by the sanitary napkin body, resulting in the dirt on the sanitary napkin being directly exposed to the air, and the reliability of use is very low.

Secondly, since one end of the fixing strap is fixed to the bottom surface of the sanitary napkin body by heat pressing, although it can achieve good connection firmness, there is no anti-tearing structure between the fixing strap and the sanitary napkin body, when the fixing strap is opened from the other end, if the user is not careful, the whole fixing strap will be easily torn off, resulting in the separation of the fixing strap and the sanitary napkin body. In this case, if continuing to bind the two ends of the fixing strap by gluing them together, it is necessary to press the two ends of the fixing strap with both hands respectively, which is very inconvenient and the firmness of the binding will also be affected. Alternatively, it is also possible to directly use a knotting method to bind the sanitary napkin, but this is very troublesome and easy to dirty your hands.

Therefore, in view of the above-mentioned deficiencies of the above-mentioned sanitary napkin products that can be rolled up, it is very necessary to redesign their structure to better meet people's application needs.

### SUMMARY

The purpose of the present application is to solve the above problems and shortcomings, and to provide a sanitary napkin structure convenient for rolling up after use. The sanitary napkin structure is provided with a second adhesive portion on the foldable binding strap, which does not contact the underwear and the sanitary napkin body, and can ensure that the second adhesive portion has excellent viscosity. After the foldable binding strap is unfolded and bound, the second adhesive portion can be adhered and fixed to the first adhesive portion. Even if the viscosity of the first adhesive portion is reduced, the second adhesive portion still has the effect of superimposing viscosity, which can firmly bind the sanitary napkin body and is not easy to be stretched. In addition, by adopting the herringbone arrangement of the second fixing portion and the first fixing portion, when the foldable binding strap is opened, the second fixing portion can provide an anti-pull force in the opposite direction, so that the foldable binding strap is not easy to be pulled off, so as to improve the connection reliability between the foldable binding strap and the sanitary napkin body, thereby ensuring that the user can perform the binding operation very conveniently, and the use is more reliable.

The technical solution of the present application is achieved as follows. A sanitary napkin structure convenient for rolling up after use includes a sanitary napkin body and a foldable binding strap. A bottom surface of the sanitary napkin body is provided with a first adhesive portion along a length direction of the sanitary napkin body, and one end of the foldable binding strap is fixed to the bottom surface of the sanitary napkin body and is connected to one end of the first adhesive portion. The foldable binding strap includes a peeling end, a first binding strap portion, a second binding strap portion, and a first fixing portion sequentially connected together, and the first fixing portion and the second binding strap portion are an integral structure. The foldable binding strap further includes a second fixing portion connected to the second binding strap portion, the second fixing portion and the first fixing portion are fixed at the bottom surface of the sanitary napkin body in a herringbone arrangement, and the first binding strap portion, the second binding strap portion, and the second fixing portion are stacked in sequence to form a three-fold state. The first binding strap portion is provided with a second adhesive portion adhered to the second binding strap portion, and the second binding strap portion is also provided with the second adhesive portion adhered to the second fixing portion.

In an embodiment, a color of the peeling end is different from a color of the first binding strap portion to form an identification structure.

In an embodiment, one end of the peeling end protrudes beyond a side edge of the sanitary napkin body.

In an embodiment, the first strap portion, the second strap portion, the first fixing portion and the second fixing portion are all strip-shaped sheet-shaped tape structures.

In an embodiment, the number of the first adhesive portion is one or more than two.

In an embodiment, the number of the first adhesive portion is four, and the four first adhesive portions are provided in parallel and at intervals; one end of two of the first adhesive portions are respectively adhered to the second fixing portion, and one end of the other two first adhesive portions are respectively provided at left and right sides of the foldable binding strap.

In an embodiment, a width B of the foldable binding strap is greater than a width C of the first adhesive portion.

In an embodiment, the width B of the foldable binding strap is 10 mm to 20 mm, and the width C of the first adhesive portion is 3 mm to 9 mm.

In an embodiment, a length D where the second fixing portion contacts the sanitary napkin body is greater than a length E where the first fixing portion contacts the sanitary napkin body.

In an embodiment, a length F of the foldable binding strap in a folded state is 40 mm to 80 mm, and a length G of the foldable binding strap in an unfolded state is 100 mm to 180 mm.

The beneficial effects of the present application are as follows: the present application adopts a foldable binding strap and a structural design that connects one end of the foldable binding strap with one end of the first adhesive portion, so that the second adhesive portion does not contact the underwear and the sanitary napkin body, does not stick to the fluff, and ensures that the second adhesive portion has excellent viscosity. After the foldable binding strap is unfolded and bound, the second adhesive portion can be adhered and fixed to the first adhesive portion. Even if the first adhesive portion is adhered to the underwear and the viscosity is reduced, the second adhesive portion still has the effect of superimposing the viscosity, so that the sanitary napkin body can be firmly bound and not easily stretched. Moreover, through such a design, it can be ensured that the first adhesive portion of the sanitary napkin body has a sufficient length to be adhered to the underwear, so as to ensure the firmness of the connection between the sanitary napkin body and the underwear. In addition, by adopting a structural design in which the second fixing portion and the first fixing portion are arranged in a herringbone shape and fixed on the bottom surface of the sanitary napkin body, when the foldable binding strap is opened, the second fixing portion can provide an anti-pulling force in the opposite direction, so that the foldable binding strap is not easily pulled off, thereby improving the connection reliability between the foldable binding strap and the sanitary napkin body, thereby ensuring that the user can perform the binding operation very conveniently and use it more reliably.

### BRIEF DESCRIPTION OF THE DRAWINGS

FIG. 1 is a schematic structural diagram of a foldable binding strap on a bottom surface of a sanitary napkin body according to the present application.

FIG. 2 is a schematic structural diagram of the foldable binding strap in the unfolded state in FIG. 1 of the present application.

FIG. 3 is one schematic structural diagram of the foldable binding strap in the unfolded state along the A-A section direction in FIG. 1 of the present application.

FIG. 4 is another schematic structural diagram of the foldable binding strap in the unfolded state along the A-A section direction in FIG. 1 of the present application.

FIG. 5 is a three-dimensional schematic diagram of a sanitary napkin structure in a bundled state according to the present application.

### DETAILED DESCRIPTION OF THE EMBODIMENTS

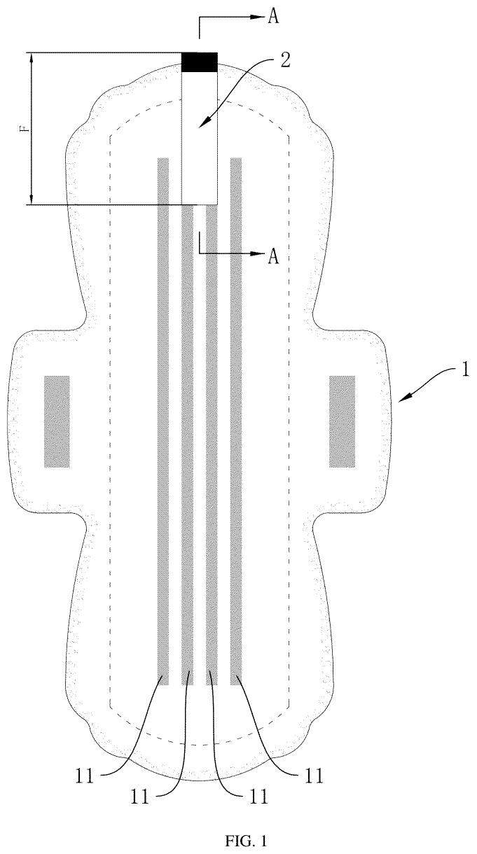

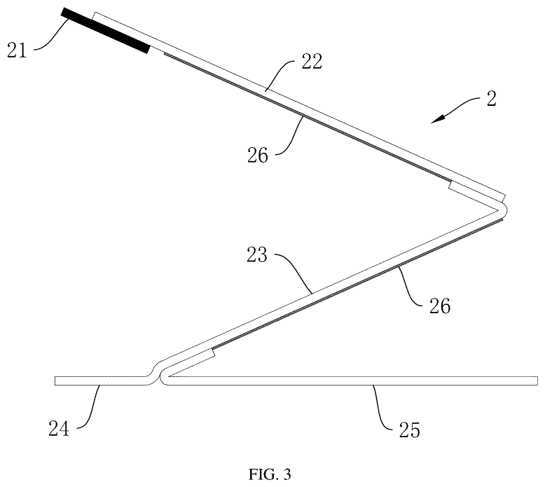

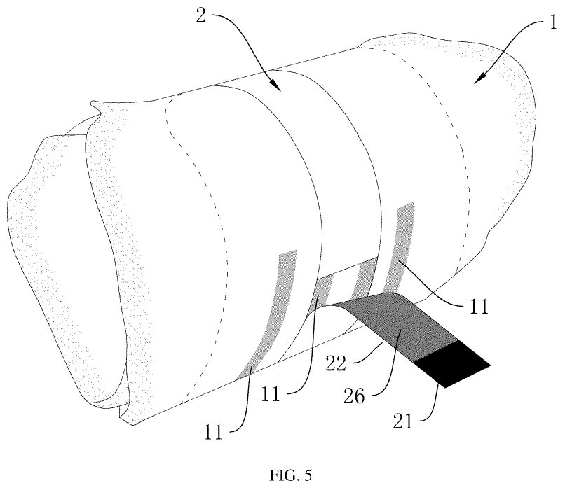

As shown in FIG. 1 and FIG. 3, the present application discloses a sanitary napkin structure convenient for rolling up after use, including a sanitary napkin body 1 and a foldable binding strap 2. A bottom surface of the sanitary napkin body 1 is provided with a first adhesive portion 11 arranged along a length direction of the sanitary napkin body 1, one end of the foldable binding strap 2 is fixed to the bottom surface of the sanitary napkin body 1, and one end of the foldable binding strap 2 is connected to one end of the first adhesive portion 11. The foldable binding strap 2 includes a peeling end 21, a first binding strap portion 22, a second binding strap portion 23, and a first fixing portion 24 that are connected in sequence, and the first fixing portion 24 and the second binding strap portion 23 are an integral structure. The foldable binding strap 2 further includes a second fixing portion 25 connected to the second binding strap portion 23, the second fixing portion 25 and the first fixing portion 24 are fixed on the bottom surface of the sanitary napkin body 1 in a herringbone arrangement, and the first binding strap portion 22, the second binding strap portion 23 and the second fixing portion 25 are stacked in sequence into a three-fold state. The first binding strap portion 22 is provided with a second adhesive portion 26 adhered to the second binding strap portion 23, and the second binding strap portion 23 is also provided with a second adhesive portion 26 adhered to the second fixing portion 25. As shown in FIG. 5, the present application provides a second adhesive portion 26 on the foldable binding strap 2, which does not contact the underwear and the sanitary napkin body 1, and can ensure that the second adhesive portion 26 has excellent viscosity. After the foldable binding strap 2 is unfolded and bound, the second adhesive portion 26 can be adhered and fixed to the first adhesive portion 11. Even if the viscosity of the first adhesive portion 11 is reduced, the second adhesive portion 26 can still play a role of superimposed viscosity, and the sanitary napkin body 1 can be firmly bound and not easily stretched. In addition, by adopting the herringbone arrangement design of the second fixing portion 25 and the first fixing portion 24, when the foldable binding strap 2 is uncovered, the second fixing portion 25 can provide an anti-pull force in the opposite direction, so that the foldable binding strap 2 is not easy to be pulled off, so as to improve the connection reliability between the foldable binding strap 2 and the sanitary napkin body 1, thereby ensuring that the user can perform the binding operation very conveniently and use it more reliably.

In order to more quickly identify the location of the peeling end 21, as shown in FIG. 1, the color of the peeling end 21 is different from the color of the first binding strap portion 22 to form a marking structure. This makes it easier for users to quickly find the location of the peeling end 21.

In order to more quickly, easily and accurately pinch the peeling end 21, as shown in FIG. 1, one end of the peeling end 21 is arranged to protrude from the side edge of the sanitary napkin body 1. By making the peeling end 21 protrude, the peeling end 21 can be pinched more accurately, and it is not easy to pinch the peeling end 21 together with the sanitary napkin body 1, which is more convenient to use.

In order to further improve the structure of the foldable binding strap 2, the first binding strap portion 22, the second binding strap portion 23, the first fixing portion 24 and the second fixing portion 25 are all strip-shaped sheet-shaped tape structures. The tape structure can be a transparent, semi-transparent or non-transparent tape structure. The peeling end 21 can be a sheet-shaped tape structure, a paper structure or a cloth structure.

As shown in FIG. 1, the number of the first adhesive portions 11 is one or more than two. In an embodiment, the number of the first adhesive portions 11 is four, and the four first adhesive portions 11 are arranged in parallel and spaced apart, one end of two first adhesive portions 11 are respectively adhered to the second fixing portion 25, and one end of the other two first adhesive portions 11 are respectively placed on the left and right sides of the foldable binding strap 2. By providing four first adhesive portions 11, it can be ensured that the sanitary napkin body 1 can be firmly adhered to the underwear. And by providing the first adhesive portions 11 on both sides of the foldable binding strap 2, after the sanitary napkin body 1 is adhered to the underwear, it can play a role of limiting and clamping the foldable binding strap 2, preventing the foldable binding strap 2 from loosening and unfolding, so as to avoid the foldable binding strap 2 from contacting with human skin and causing discomfort, and can also ensure that the second adhesive portion 26 has excellent viscosity, which is more reliable to use. By adhering one end of the two first adhesive portions 11 to the second fixing portion 25 respectively, the connection between the second fixing portion 25 and the sanitary napkin body 1 can be strengthened to prevent loosening.

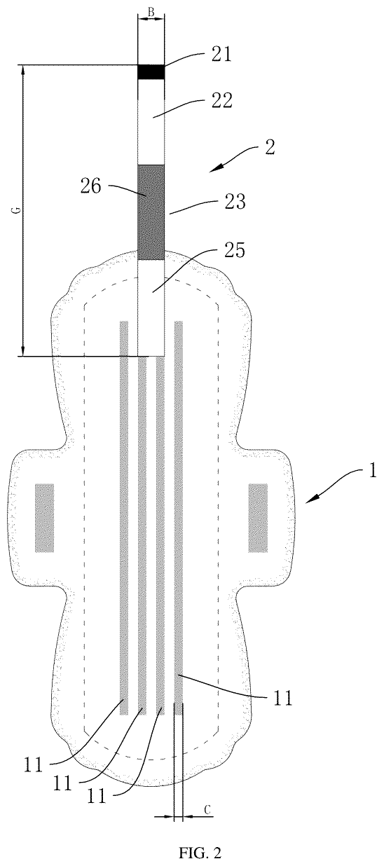

As shown in FIG. 2, the width B of the foldable binding strap 2 is greater than the width C of the first adhesive portion 11. This not only ensures that the foldable binding strap 2 is firmly connected to the sanitary napkin body 1, but also ensures that the foldable binding strap 2 has very good structural strength and is not easily torn apart. Specifically, as shown in FIG. 2, the width B of the foldable binding strap 2 is 10 mm to 20 mm. In an embodiment, the width B of the foldable binding strap 2 is 13 mm. This ensures that the width of the foldable binding strap 2 is neither too large nor too small, ensuring that the foldable binding strap 2 as a whole has very good structural strength. The width C of the first adhesive portion 11 is 3 mm to 9 mm. In an embodiment, the width C of the first adhesive portion 11 is 5 mm. This ensures that the first adhesive portion 11 has very good adhesion and is easy to pull apart when separated from the underwear.

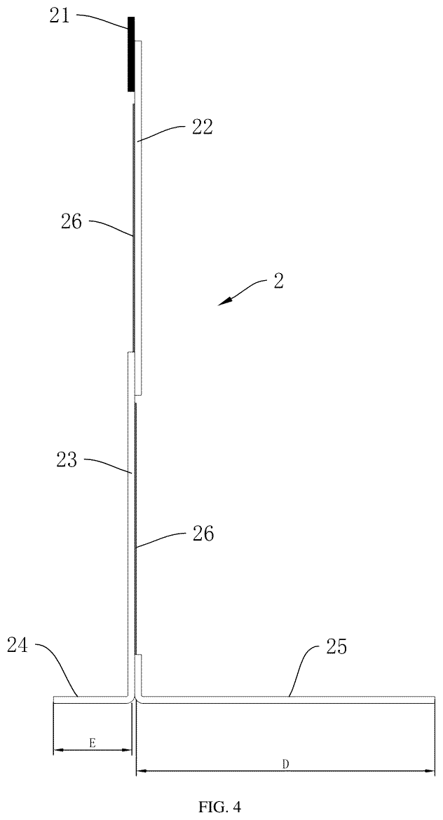

As shown in FIG. 4, the length D where the second fixing portion 25 contacts the sanitary napkin body 1 is greater than the length E where the first fixing portion 24 contacts the sanitary napkin body 1. With such a structural design, not only can the second fixing portion 25 have a sufficient contact area with the sanitary napkin body 1, so that the second fixing portion 25 can be firmly fixed to the sanitary napkin body 1 to prevent the second fixing portion 25 from being pulled off, but also ensure that the second fixing portion 25 can provide a sufficient strength of anti-pulling force in the reverse direction, thereby ensuring that the foldable binding strap 2 is not easily pulled off and is not easily separated from the sanitary napkin body 1.

As shown in FIG. 1 and FIG. 2, the length F of the foldable binding strap 2 in the folded state is 40 mm to 80 mm. In an embodiment, the length F of the foldable binding strap 2 in the folded state is 63 mm. In this way, the foldable binding strap 2 can be firmly connected to the sanitary napkin body 1 without occupying too much of the length set by the first adhesive portion 11, and has enough length to bind the sanitary napkin body 1 after unfolding. The length G of the foldable binding strap 2 in the unfolded state is 100 mm to 180 mm. In an embodiment, the length G of the foldable binding strap 2 in the unfolded state is 142 mm. In this way, it can be ensured that the foldable binding strap 2 has enough length to bind the sanitary napkin body 1 after unfolding.

In actual application, the first fixing portion 24 and the second fixing portion 25 are fixed to the bottom surface of the sanitary napkin body 1 by gluing, which not only ensures the firmness of the connection but also simplifies the manufacturing process.

What is claimed is:

1. A sanitary napkin structure convenient for rolling up after use, comprising:
a sanitary napkin body; and
a foldable binding strap,
wherein a bottom surface of the sanitary napkin body is provided with a first adhesive portion along a length direction of the sanitary napkin body, and one end of the foldable binding strap is fixed to the bottom surface of the sanitary napkin body and is connected to one end of the first adhesive portion;
the foldable binding strap comprises a peeling end, a first binding strap portion, a second binding strap portion, and a first fixing portion sequentially connected together, and the first fixing portion and the second binding strap portion are an integral structure;
the foldable binding strap further comprises a second fixing portion connected to the second binding strap portion, the second fixing portion and the first fixing portion are fixed at the bottom surface of the sanitary napkin body in a herringbone arrangement, and the first binding strap portion, the second binding strap portion, and the second fixing portion are stacked in sequence to form a three-fold state; and
the first binding strap portion is provided with a second adhesive portion adhered to the second binding strap portion, and the second binding strap portion is also provided with the second adhesive portion adhered to the second fixing portion.

2. The sanitary napkin structure convenient for rolling up after use according to claim 1, wherein a color of the peeling end is different from a color of the first binding strap portion to form an identification structure.

3. The sanitary napkin structure convenient for rolling up after use according to claim 1, wherein one end of the peeling end protrudes beyond a side edge of the sanitary napkin body.

4. The sanitary napkin structure convenient for rolling up after use according to claim 1, wherein the first strap portion, the second strap portion, the first fixing portion and the second fixing portion are all strip-shaped sheet-shaped tape structures.

5. The sanitary napkin structure convenient for rolling up after use according to claim 1, wherein the number of the first adhesive portion is one or more than two.

6. The sanitary napkin structure convenient for rolling up after use according to claim 5, wherein the number of the first adhesive portion is four, and the four first adhesive portions are provided in parallel and at intervals; one end of two of the first adhesive portions are respectively adhered to the second fixing portion, and one end of the other two first adhesive portions are respectively provided at left and right sides of the foldable binding strap.

7. The sanitary napkin structure convenient for rolling up after use according to claim 1, wherein a width of the foldable binding strap is greater than a width of the first adhesive portion.

8. The sanitary napkin structure convenient for rolling up after use according to claim 7, wherein the width of the foldable binding strap is 10 mm to 20 mm, and the width of the first adhesive portion is 3 mm to 9 mm.

9. The sanitary napkin structure convenient for rolling up after use according to claim 1, wherein a length where the second fixing portion contacts the sanitary napkin body is greater than a length where the first fixing portion contacts the sanitary napkin body.

10. The sanitary napkin structure convenient for rolling up after use according to claim 1, wherein a length of the foldable binding strap in a folded state is 40 mm to 80 mm, and a length of the foldable binding strap in an unfolded state is 100 mm to 180 mm.
